(19) United States
(12) Patent Application Publication
(10) Pub. No.: US 2011/0257454 A1
(43) Pub. Date: Oct. 20, 2011

(54) Use of an Additive in the Coupling of Toluene with a Carbon Source

(75) Inventors: Joseph L. Thorman, Milwaukee, WI (US); Sivadinarayana Chinta, Missouri City, TX (US); James Butler, League City, TX (US)

(73) Assignee: Fina Technology, Inc., Houston, TX (US)

(21) Appl. No.: 12/938,449

(22) Filed: Nov. 3, 2010

Related U.S. Application Data
(63) Continuation-in-part of application No. 12/763,234, filed on Apr. 20, 2010.

(51) Int. Cl. C07C 15/46; B01J 29/08; B01J 29/14; B01J 37/30

(52) U.S. Cl. 585/438; 502/79; 502/61; 502/74

(57) ABSTRACT

A method is disclosed of preparing a catalyst including providing a substrate and a first solution containing at least one promoter, contacting the substrate with the solution to obtain a catalyst containing at least one promoter, wherein the contacting of the substrate with the solution subjects the substrate to the addition of at least one promoter.

### CROSS-REFERENCE TO RELATED APPLICATIONS

The present invention is a continuation-in-part (CIP) of U.S. patent application Ser. No. 12/763,234 filed by Fina Technology, Inc. on Apr. 20, 2010.

### FIELD

The present invention relates to a method for the production of styrene and ethylbenzene. More specifically, the invention relates to the alkylation of toluene with a carbon source (herein referred to as a C₁ source) such as methanol and/or formaldehyde, to produce styrene and ethylbenzene.

### BACKGROUND

Styrene is an important monomer used in the manufacture of many plastics. Styrene is commonly produced by making ethylbenzene, which is then dehydrogenated to produce styrene. Ethylbenzene is typically formed by one or more aromatic conversion processes involving the alkylation of benzene.

Aromatic conversion processes, which are typically carried out utilizing a molecular sieve type catalyst, are well known in the chemical processing industry. Such aromatic conversion processes include the alkylation of aromatic compounds such as benzene with ethylene to produce alkyl aromatics such as ethylbenzene. Typically an alkylation reactor, which can produce a mixture of monoalkyl and polyalkyl benzenes, will be coupled with a transalkylation reactor for the conversion of polyalkyl benzenes to monoalkyl benzenes. The transalkylation process is operated under conditions to cause disproportionation of the polyalkylated aromatic fraction, which can produce a product having an enhanced ethylbenzene content and reduced polyalkylated content. When both alkylation and transalkylation processes are used, two separate reactors, each with its own catalyst, can be employed for each of the processes.

Ethylene is obtained predominantly from the thermal cracking of hydrocarbons, such as ethane, propane, butane, or naphtha. Ethylene can also be produced and recovered from various refinery processes. Thermal cracking and separation technologies for the production of relatively pure ethylene can account for a significant portion of the total ethylbenzene production costs.

Benzene can be obtained from the hydrodealkylation of toluene that involves heating a mixture of toluene with excess hydrogen to elevated temperatures (for example 500° C. to 600° C.) in the presence of a catalyst. Under these conditions, toluene can undergo dealkylation according to the chemical equation: C₆H₅CH₃+H₂→C₆H₆+CH₄. This reaction requires energy input and as can be seen from the above equation, produces methane as a byproduct, which is typically separated and may be used as heating fuel for the process.

Another known process includes the alkylation of toluene to produce styrene and ethylbenzene. In this alkylation process, various aluminosilicate catalysts are utilized to react methanol and toluene to produce styrene and ethylbenzene. However, such processes have been characterized by having very low yields in addition to having very low selectivity to styrene and ethylbenzene.

Also, the aluminosilicate catalysts are typically prepared using solutions of acetone and other highly flammable organic substances, which can be hazardous and require additional drying steps. For instance a typical aluminosilicate catalyst can include various promoters supported on a zeolitic substrate. These catalysts can be prepared by subjecting the zeolite to an ion-exchange in an aqueous solution followed by a promoter metal impregnation using acetone. This method requires an intermediate drying step after the ion-exchange to remove all water prior to the promoter metal impregnation with acetone. After the promoter metal impregnation the catalyst is subjected to a further drying step to remove all acetone. This intermediate drying step typically involves heating to at least 150° C., which results in increased costs.

In view of the above, it would be desirable to have a process of producing styrene and/or ethylbenzene that does not rely on thermal crackers and expensive separation technologies as a source of ethylene. It would further be desirable to avoid the process of converting toluene to benzene with its inherent expense and loss of a carbon atom to form methane. It would be desirable to produce styrene without the use of benzene and ethylene as feedstreams. It would also be desirable to produce styrene and/or ethylbenzene in one reactor without the need for separate reactors requiring additional separation steps. Furthermore, it is desirable to achieve a process having a high yield and selectivity to styrene and ethylbenzene. Even further, it is desirable to achieve a process having a high yield and selectivity to styrene such that the step of dehydrogenation of ethylbenzene to produce styrene can be reduced. It is further desirable to be able to produce a catalyst having the properties desired without involving flammable materials and/or intermediate drying steps.

### SUMMARY

An embodiment of the present invention is a method of preparing a catalyst by providing a substrate and a first solution comprising at least one promoter and contacting the substrate with the first solution to obtain a catalyst comprising at least one promoter. The contacting of the substrate with the solution subjects the substrate to ion exchange wherein cationic sites on the substrate are exchanged for the at least one promoter. The substrate can be a zeolite. The promoter(s) can be selected from the group consisting of Ru, Rh, Ni, Co, Pd, Pt, Mn, Ti, Zr, V, Nb, K, Cs, Ga, B, P, Rb, Ag, Na, Cu, Mg, and combinations thereof.

The method can include a second solution that includes Cs and the promoter of the first solution includes B. The first and second solutions can contact the substrate resulting in a substrate comprising B and Cs.

The method can include a second solution that includes Cs and the promoter of the first solution includes B. The first solution can initially contact the substrate resulting in a substrate including B, followed by contacting the substrate comprising B with the second solution comprising Cs resulting in a substrate comprising B and Cs.

The catalyst can have B in amounts ranging from 0.1 wt % to 3 wt % based on the total weight of the catalyst, as determined by elemental analysis. The B in the first solution can be supplied by a boron source comprising boroxines. The catalyst can be capable of effecting a reaction of at least a portion of a C₁ source with toluene to form a product stream comprising one or more of styrene or ethylbenzene and capable of effecting a toluene conversion of greater than 0.1 mol %. A boron source can be combined with the substrate prior to contacting the substrate with the first solution. The boron source can be combined with a substrate material that is subsequently combined with the catalyst comprising at least one promoter to form a supported catalyst comprising at least one promoter.

An alternate embodiment is a catalyst having a zeolitic support, at least one promoter selected from the group consisting of Cs, B, Ga, Rb, K, and combinations thereof. The promoter(s) can be supported onto the zeolitic support by ion exchange, or by another mechanism. The promoter(s) can contain B obtained from a boron source such as boroxines. The promoter(s) can include a combination of Cs and B. The ion exchange can be performed in an aqueous medium utilizing water soluble promoter precursors. The boron can be present in the catalyst in amounts of from 0.1 to 3 wt % based on the total weight of the catalyst.

A boron source can be combined with a substrate material that is subsequently combined with the zeolitic support having at least one promoter to form a supported catalyst with at least one promoter. The catalyst can be capable of effecting a reaction of at least a portion of a C₁ source with toluene to form a product stream having styrene or ethylbenzene, wherein the catalyst is capable of effecting selectivity to styrene of greater than 30 mol %.

A further embodiment of the invention is a process for making styrene by providing a C₁ source to a reactor having a catalyst that includes B and Cs supported on a zeolite. Toluene is reacted with the C₁ source in the presence of the catalyst to form a product stream having ethylbenzene and styrene. The C₁ source can be selected from the group consisting of methanol, formaldehyde, formalin, trioxane, methylformcel, paraformaldehyde, methylal, and combinations thereof. The B can be present on the catalyst in amounts of up to 3 wt % based on the total weight of the catalyst and the B was supplied by a boron source comprising boroxines.

The B and Cs can be added to the zeolite by use of an aqueous medium utilizing water-soluble B and Cs precursors. The boron source can be added to the C₁ source and/or the toluene feed. The catalyst can be a supported catalyst made from a boron source combined with a substrate material that is added to the catalyst that has B and Cs supported on a zeolite. The catalyst can be capable of effecting a toluene conversion of greater than 0.1 mol %.

The various aspects of the present invention can be joined in combination with other aspects of the invention and the listed embodiments herein are not meant to limit the invention. All combinations of aspects of the invention are enabled, even if not given in a particular example herein.

### BRIEF DESCRIPTION OF DRAWINGS

FIG. 1 illustrates a flow chart for the production of styrene by the reaction of formaldehyde and toluene, wherein the formaldehyde is first produced in a separate reactor by either the dehydrogenation or oxidation of methanol and is then reacted with toluene to produce styrene.

FIG. 2 illustrates a flow chart for the production of styrene by the reaction of formaldehyde and toluene, wherein methanol and toluene are fed into a reactor, wherein the methanol is converted to formaldehyde and the formaldehyde is reacted with toluene to produce styrene.

FIG. 3 depicts a graph showing the effect of boron weight percent on toluene conversion.

FIG. 4 depicts a graph showing the effect of boron weight percent on styrene selectivity.

### DETAILED DESCRIPTION

In an aspect of the current invention, toluene is reacted with a carbon source capable of coupling with toluene to form ethylbenzene or styrene, which can be referred to as a C₁ source, to produce styrene and ethylbenzene. In an embodiment, the C₁ source includes methanol or formaldehyde or a mixture of the two. In an alternative embodiment, toluene is reacted with one or more of the following: formalin, trioxane, methylformcel, paraformaldehyde and methylal. In a further embodiment, the C₁ source is selected from the group consisting of methanol, formaldehyde, formalin (37-50% H₂CO in solution of water and MeOH), trioxane (1,3,5-trioxane), methylformcel (55% H₂CO in methanol), paraformaldehyde and methylal (dimethoxymethane), and combinations thereof.

Formaldehyde can be produced either by the oxidation or dehydrogenation of methanol.

In an embodiment, formaldehyde is produced by the dehydrogenation of methanol to produce formaldehyde and hydrogen gas. This reaction step produces a dry formaldehyde stream that may be preferred, as it would not require the separation of the water prior to the reaction of the formaldehyde with toluene. The dehydrogenation process is described in the equation below:

CH₃OH→CH₂O+H₂

Formaldehyde can also be produced by the oxidation of methanol to produce formaldehyde and water. The oxidation of methanol is described in the equation below:

2CH₃OH+O₂→2CH₂O+2H₂O

In the case of using a separate process to obtain formaldehyde, a separation unit may then be used in order to separate the formaldehyde from the hydrogen gas or water from the formaldehyde and unreacted methanol prior to reacting the formaldehyde with toluene for the production of styrene. This separation would inhibit the hydrogenation of the formaldehyde back to methanol. Purified formaldehyde could then be sent to a styrene reactor and the unreacted methanol could be recycled.

Although the reaction has a 1:1 molar ratio of toluene and the C₁ source, the ratio of the feedstreams is not limited within the present invention and can vary depending on operating conditions and the efficiency of the reaction system. If excess toluene or C₁ source is fed to the reaction zone, the unreacted portion can be subsequently separated and recycled back into the process. In one embodiment the ratio of toluene:C₁ source can range from between 100:1 to 1:100. In alternate embodiments the ratio of toluene:C₁ source can range from 50:1 to 1:50; from 20:1 to 1:20; from 10:1 to 1:10; from 5:1 to 1:5; from 2:1 to 1:2. In a specific aspect, the ratio of toluene:C₁ source can range from 2:1 to 5:1.

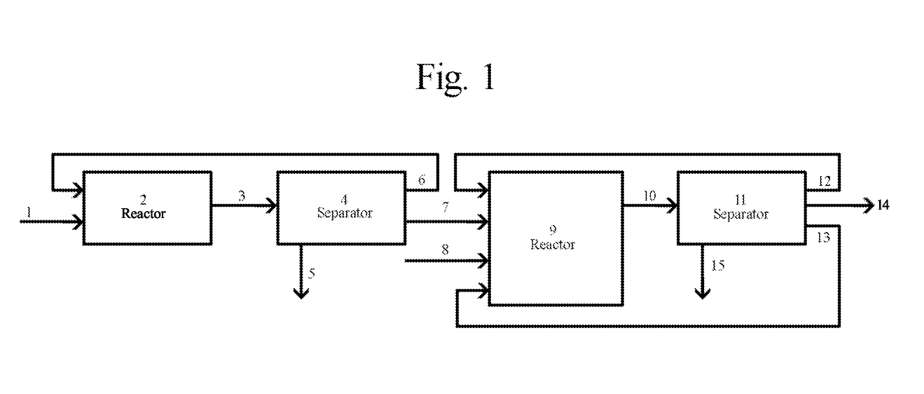

In FIG. 1 there is a simplified flow chart of one embodiment of the styrene production process described above. In this embodiment, a first reactor (2) is either a dehydrogenation reactor or an oxidation reactor. This reactor is designed to convert the first methanol feed (1) into formaldehyde. The gas product (3) of the reactor is then sent to a gas separation unit (4) where the formaldehyde is separated from any unreacted methanol and unwanted byproducts. Any unreacted methanol (6) can then be recycled back into the first reactor (2). The byproducts (5) are separated from the clean formaldehyde (7).

In one embodiment the first reactor (2) is a dehydrogenation reactor that produces formaldehyde and hydrogen and the separation unit (4) is a membrane capable of removing hydrogen from the product stream (3).

In an alternate embodiment the first reactor (2) is an oxidative reactor that produces product stream (3) comprising formaldehyde and water. The product stream (3) comprising formaldehyde and water can then be sent to the second reactor (9) without a separation unit (4).

The formaldehyde feed stream (7) is then reacted with a feed stream of toluene (8) in a second reactor (9). The toluene and formaldehyde react to produce styrene. The product (10) of the second reactor (9) may then be sent to an optional separation unit (11) where any unwanted byproducts (15) such as water can separated from the styrene, unreacted formaldehyde and unreacted toluene. Any unreacted formaldehyde (12) and the unreacted toluene (13) can be recycled back into the reactor (9). A styrene product stream (14) can be removed from the separation unit (11) and subjected to further treatment or processing if desired.

The operating conditions of the reactors and separators will be system specific and can vary depending on the feedstream composition and the composition of the product streams. The reactor (9) for the reaction of toluene and formaldehyde will operate at elevated temperatures and pressures and may contain a basic or neutral catalyst system. The temperature can range in a non-limiting example from 250° C. to 750° C., optionally from 300° C. to 500° C., optionally from 325° C. to 450° C. The pressure can range in a non-limiting example from 0.1 atm to 70 atm, optionally from 0.1 atm to 35 atm, optionally from 0.1 atm to 5 atm.

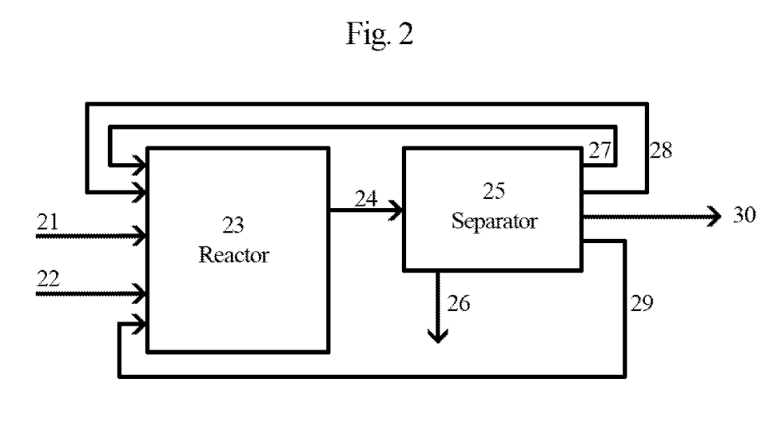

FIG. 2 is a simplified flow chart of another embodiment of the styrene process discussed above. A C₁ source containing feed stream (21) is fed along with a feed stream of toluene (22) in a reactor (23). Toluene and the C₁ source then react to produce styrene. The product (24) of the reactor (23) may then be sent to an optional separation unit (25) where any unwanted byproducts (26) can be separated from the styrene, and any unreacted C1 source, unreacted methanol, unreacted formaldehyde and unreacted toluene. Any unreacted methanol (27), unreacted formaldehyde (28) and the unreacted toluene (29) can be recycled back into the reactor (23). A styrene product stream (30) can be removed from the separation unit (25) and subjected to further treatment or processing if desired.

The operating conditions of the reactors and separators will be system specific and can vary depending on the feedstream composition and the composition of the product streams. The reactor (23) for the reactions of methanol to formaldehyde and toluene with a C₁ source, such as formaldehyde, will operate at elevated temperatures and pressures and may contain a basic or neutral catalyst system. The temperature can range in a non-limiting example from 250° C. to 750° C., optionally from 300° C. to 500° C., optionally from 325° C. to 450° C. The pressure can range in a non-limiting example from 0.1 atm to 70 atm, optionally from 0.1 atm to 35 atm, optionally from 0.1 atm to 5 atm.

Improvement in side chain alkylation selectivity may be achieved by treating a molecular sieve zeolite catalyst with chemical compounds to inhibit the external acidic sites and minimize aromatic alkylation on the ring positions. Another means of improvement of side chain alkylation selectivity can be to inhibit overly basic sites, such as for example with the addition of a boron compound. Another means of improvement of side chain alkylation selectivity can be to impose restrictions on the catalyst structure to facilitate side chain alkylation. In one embodiment the catalyst used in an embodiment of the present invention is a basic or neutral catalyst.

The catalytic reaction systems suitable for this invention can include one or more of the zeolite or amorphous materials modified for side chain alkylation selectivity. A non-limiting example can be a zeolite promoted with one or more of the following: Ru, Rh, Ni, Co, Pd, Pt, Mn, Ti, Zr, V, Nb, K, Cs, Ga, B, P, Rb, Ag, Na, Cu, Mg, or combinations thereof. In an embodiment, the zeolite can be promoted with one or more of Cs, B, Co, or Ga, or combinations thereof. In another embodiment, the zeolite can be promoted with one selected from the group of Cs, B, Ga, and K and any combinations thereof. The promoter can exchange with an element within the zeolite or amorphous material and/or be attached to the zeolite or amorphous material in an occluded manner. In an aspect the amount of promoter is determined by the amount needed to yield less than 0.5 mol % of ring alkylated products such as xylenes from a coupling reaction of toluene and a C₁ source.

In an embodiment, the catalyst contains greater than 0.1 wt % of at least one promoter based on the total weight of the catalyst. In another embodiment, the catalyst contains up to 5 wt % of at least one promoter. In a further embodiment, the catalyst contains from 0.1 to 3 wt % of at least one promoter. In an aspect, the at least one promoter is boron.

Zeolite materials suitable for this invention may include silicate-based zeolites and amorphous compounds such as faujasites, mordenites, etc. Silicate-based zeolites are made of alternating SiO₂ and MOₓ tetrahedra, where M is an element selected from the Groups 1 through 16 of the Periodic Table (new IUPAC). These types of zeolites have 4, 6, 8, 10, or 12-membered oxygen ring channels. An example of zeolites of this invention can include faujasites, such as an X-type or Y-type zeolite.

In an embodiment, the zeolite materials suitable for this invention are characterized by silica to alumina ratio (Si/Al) of less than 1.5. In another embodiment, the zeolite materials are characterized by a Si/Al ratio ranging from 1.0 to 200, optionally from 1.0 to 100, optionally from 1.0 to 50, optionally from 1.0 to 10, optionally from 1.0 to 2.0, optionally from 1.0 to 1.5.

The present catalyst is adaptable to use in the various physical forms in which catalysts are commonly used. The catalyst of the invention may be used as a particulate material in a contact bed or as a coating material on structures having a high surface area. If desired, the catalyst can be deposited with various catalyst binder and/or support materials.

A catalyst comprising a substrate that supports a promoting metal or a combination of metals can be used to catalyze the reaction of hydrocarbons. The method of preparing the catalyst, pretreatment of the catalyst, and reaction conditions can influence the conversion, selectivity, and yield of the reactions.

The various elements that make up the catalyst can be derived from any suitable source, such as in their elemental form, or in compounds or coordination complexes of an organic or inorganic nature, such as carbonates, oxides, hydroxides, nitrates, acetates, chlorides, phosphates, sulfides and sulfonates. The elements and/or compounds can be prepared by any suitable method, known in the art, for the preparation of such materials.

The term “substrate” as used herein is not meant to indicate that this component is necessarily inactive, while the other metals and/or promoters are the active species. On the contrary, the substrate can be an active part of the catalyst. The term “substrate” would merely imply that the substrate makes up a significant quantity, generally 10% or more by weight, of the entire catalyst. The promoters individually can range from 0.01% to 60% by weight of the catalyst, optionally from 0.01% to 50%, optionally from 0.01% to 40%, optionally from 0.01% to 30%, optionally from 0.01% to 20%, optionally from 0.01% to 10%, optionally from 0.01% to 5%. If more than one promoter is combined, they together generally can range from 0.01% up to 70% by weight of the catalyst, optionally from 0.01% to 50%, optionally from 0.01% to 30%, optionally from 0.01% to 15%, optionally from 0.01% to 5%. The elements of the catalyst composition can be provided from any suitable source, such as in its elemental form, as a salt, as a coordination compound, etc.

The addition of a support material to improve the catalyst physical properties is possible within the present invention. Binder material, extrusion aids or other additives can be added to the catalyst composition or the final catalyst composition can be added to a structured material that provides a support structure. For example, the final catalyst composition can include an alumina or aluminate framework as a support. Upon calcination these elements can be altered, such as through oxidation which would increase the relative content of oxygen within the final catalyst structure. The combination of the catalyst of the present invention combined with additional elements such as a binder, extrusion aid, structured material, or other additives, and their respective calcination products, are included within the scope of the invention.

In one embodiment, the catalyst can be prepared by combining a substrate with at least one promoter element. Embodiments of a substrate can be a molecular sieve, from either natural or synthetic sources. Zeolites and zeolite-like materials can be an effective substrate. Alternate molecular sieves also contemplated are zeolite-like materials such as the crystalline silicoaluminophosphates (SAPO) and the aluminophosphates (ALPO).

The present invention is not limited by the method of catalyst preparation, and all suitable methods should be considered to fall within the scope herein. Particularly effective techniques are those utilized for the preparation of solid catalysts. Conventional methods include co-precipitation from an aqueous, an organic or a combination solution-dispersion, impregnation, dry mixing, wet mixing or the like, alone or in various combinations. In general, any method can be used which provides compositions of matter containing the prescribed components in effective amounts. According to an embodiment the substrate is charged with promoter via an incipient wetness impregnation. Other impregnation techniques such as by soaking, pore volume impregnation, or percolation can optionally be used. Alternate methods such as ion exchange, wash coat, precipitation, and gel formation can also be used. Various methods and procedures for catalyst preparation are listed in the technical report Manual of Methods and Procedures for Catalyst Characterization by J. Haber, J. H. Block and B. Dolmon, published in the International Union of Pure and Applied Chemistry, Volume 67, Nos 8/9, pp. 1257-1306, 1995, incorporated herein in its entirety.

The promoter elements can be added to or incorporated into the substrate in any appropriate form. In an embodiment, the promoter elements are added to the substrate by mechanical mixing, by impregnation in the form of solutions or suspensions in an appropriate liquid, or by ion exchange. In a more specific embodiment, the promoter elements are added to the substrate by impregnation in the form of solutions or suspensions in a liquid selected from the group of acetone, anhydrous (or dry) acetone, methanol, and aqueous solutions.

In another more specific embodiment, the promoter is added to the substrate by ion exchange. Ion exchange may be performed by conventional ion exchange methods in which sodium, hydrogen, or other inorganic cations that may be typically present in a substrate are at least partially replaced via a fluid solution. In an embodiment, the fluid solution can include any medium that will solubilize the cation without adversely affecting the substrate. In an embodiment, the ion exchange is performed by heating a solution containing any promoter selected from the group of Ru, Rh, Ni, Co, Pd, Pt, Mn, Ti, Zr, V, Nb, K, Cs, Ga, P, Rb, Ag, Na, Cu, Mg, and any combinations thereof in which the promoter(s) is(are) solubilized in the solution, which may be heated, and contacting the solution with the substrate. In another embodiment, the ion exchange includes heating a solution containing any one selected from the group of Cs, Ga, Rb, and K and any combinations thereof. In an embodiment, the solution is heated to temperatures ranging from 50 to 120° C. In another embodiment, the solution is heated to temperatures ranging from 80 to 100° C.

The solution for use in the ion exchange method may include any fluid medium. A non-fluid ion exchange is also possible and within the scope of the present invention. In an embodiment, the solution for use in the ion exchange method includes an aqueous medium or an organic medium. In a more specific embodiment, the solution for use in the ion exchange method includes water.

The promoters may be incorporated into the substrate in any order or arrangement. In an embodiment, all of the promoters are simultaneously incorporated into the substrate. In more specific embodiment, each promoter is in an aqueous solution for ion-exchange with and/or impregnation to the substrate. In another embodiment, each promoter is in a separate aqueous solution, wherein each solution is simultaneously contacted with the substrate for ion-exchange with and/or impregnation to the substrate. In a further embodiment, each promoter is in a separate aqueous solution, wherein each solution is separately contacted with the substrate for ion-exchange with and/or impregnation to the substrate.

In an aspect, the at least one promoter includes boron. In an embodiment, the catalyst contains greater than 0.1 wt % boron based on the total weight of the catalyst. In another embodiment, the catalyst contains from 0.1 to 3 wt % boron.

The boron promoter can be added to the catalyst by contacting the substrate, impregnation, or any other method, with any known boron source. In an embodiment, the boron source is selected from the group of boric acid, boron phosphate, methoxyboroxine, methylboroxine, and trimethoxyboroxine and combinations thereof. In another embodiment, the boron source contains boroxines. In a further embodiment, the boron source is selected from the group of methoxyboroxine, methylboroxine, and trimethoxyboroxine and combinations thereof.

In an embodiment, a substrate may be previously treated with a boron source prior to an addition of at least one promoter, wherein the at least one promoter includes boron. In another embodiment, a boron treated zeolite may be combined with at least one promoter, wherein the at least one promoter includes boron. In a further embodiment, boron may be added to the catalyst system by adding at least one promoter containing boron as a co-feed with toluene and methanol. In an even further embodiment, boron may be added to the catalyst system by adding boroxines as a co-feed with toluene and methanol. The boroxines can include, methoxyboroxine, methylboroxine, and trimethoxyboroxine, and combinations thereof. The boron treated zeolite further combined with at least one promoter including boron may be used in preparing a supported catalyst such as extrudates and tablets.

When slurries, precipitates or the like are prepared, they may be dried, usually at a temperature sufficient to volatilize the water or other carrier, such as from 100° C. to 250° C., with or without vacuum. Irrespective of how the components are combined and irrespective of the source of the components, the dried composition is generally calcined in the presence of an oxygen-containing gas, usually at temperatures between about 300° C. and about 900° C. for from 1 to 24 hours. The calcination can be in an oxygen-containing atmosphere, or alternately in a reducing or inert atmosphere.

The prepared catalyst can be ground, pressed, sieved, shaped and/or otherwise processed into a form suitable for loading into a reactor. The reactor can be any type known in the art to make catalyst particles, such as a fixed bed, fluidized bed, or swing bed reactor. Optionally an inert material, such as quartz chips, can be used to support the catalyst bed and to place the catalyst within the bed. Depending on the catalyst, a pretreatment of the catalyst may, or may not, be necessary. For the pretreatment, the reactor can be heated to elevated temperatures, such as 200° C. to 900° C. with an air flow, such as 100 mL/min, and held at these conditions for a length of time, such as 1 to 3 hours. Then, the reactor can be brought to the operating temperature of the reactor, for example 300° C. to 550° C., or optionally down to any desired temperature, for instance down to ambient temperature to remain under a purge until it is ready to be put in service. The reactor can be kept under an inert purge, such as under a nitrogen or helium purge.

Embodiments of reactors that can be used with the present invention can include, by non-limiting examples: fixed bed reactors; fluid bed reactors; and entrained bed reactors. Reactors capable of the elevated temperature and pressure as described herein, and capable of enabling contact of the reactants with the catalyst, can be considered within the scope of the present invention. Embodiments of the particular reactor system may be determined based on the particular design conditions and throughput, as by one of ordinary skill in the art, and are not meant to be limiting on the scope of the present invention. An example of a suitable reactor can be a fluid bed reactor having catalyst regeneration capabilities. This type of reactor system employing a riser can be modified as needed, for example by insulating or heating the riser if thermal input is needed, or by jacketing the riser with cooling water if thermal dissipation is required. These designs can also be used to replace catalyst while the process is in operation, by withdrawing catalyst from the regeneration vessel from an exit line or adding new catalyst into the system while in operation.

In another aspect, the one or more reactors may include one or more catalyst beds. In the event of multiple beds, an inert material layer can separate each bed. The inert material can comprise any type of inert substance, including quartz. In an embodiment, a reactor includes between 1 and 25 catalyst beds. In a further embodiment, a reactor includes between 2 and 10 catalyst beds. In a further embodiment, a reactor includes between 2 and 5 catalyst beds. In addition, the C₁ source and toluene may be injected into a catalyst bed, an inert material layer, or both. In a further embodiment, at least a portion of the C₁ source is injected into a catalyst bed(s) and at least a portion of the toluene feed is injected into an inert material layer(s).

In an alternate embodiment, the entire C₁ source is injected into a catalyst bed(s) and all of the toluene feed is injected into an inert material layer(s). In another aspect, at least a portion of the toluene feed is injected into a catalyst bed(s) and at least a portion the C₁ source is injected into an inert material layer(s). In a further aspect, all of the toluene feed is injected into a catalyst bed(s) and the entire C₁ source is injected into an inert material layer(s).

The toluene and C₁ source coupling reaction may have a toluene conversion percent greater than 0.01 mol %. In an embodiment the toluene and C₁ source coupling reaction is capable of having a toluene conversion percent in the range of from 0.05 mol % to 40 mol %. In a further embodiment the toluene and C₁ source coupling reaction is capable of having a toluene conversion in the range of from 2 mol % to 40 mol %, optionally from 5 mol % to 35 mol %, optionally from 20 mol % to 30 mol %.

In an aspect the toluene and C₁ source coupling reaction is capable of selectivity to styrene greater than 1 mol %. In another aspect, the toluene and C₁ source coupling reaction is capable of selectivity to styrene in the range of from 1 mol % to 99 mol %. In an aspect the toluene to a C₁ source coupling reaction is capable of selectivity to ethylbenzene greater than 1 mol %. In another aspect, the toluene and C₁ source coupling reaction is capable of selectivity to ethylbenzene in the range of from 1 mol % to 99 mol %. In an aspect the toluene and C₁ source coupling reaction is capable of yielding less than 0.5 mol % of ring alkylated products such as xylenes.

### EXAMPLES

### Example 1

Procedure used to produce the cesium ion-exchanged X-zeolite material: A glass cylinder (2″ inside diameter), fitted with a sintered glass disk and stopcock at the lower end, was charged with 544-HP zeolite (100 g, W.C. Grace) and CsOH (400 mL, 1.0 M in water). The mixture was then brought to 90° C. and allowed to stand for 4 h. The liquid was drained from the zeolite material and another aliquot of CsOH (400 mL of 1.0 M solution in water) was added, heated, and allowed to stand for 3 hours at 90° C. The liquid was drained from the zeolite material and another aliquot of CsOH (400 mL of 1.0 M solution in water) was added, heated, and allowed to stand for 15 hours at 90° C. The liquid was drained from the zeolite material and dried at 150° C. for 1.5 hours.

Incipient wetness impregnation of Ga(NO₃)₃ on to the cesium ion-exchanged X-zeolite material: The cesium ion-exchanged zeolite material (50 g) was subjected to incipient wetness impregnation of Ga(NO₃)₃ by adding the Ga(NO₃)₃ solution (1.83 g Ga(NO₃)₃ in 13.3 mL of water) to the zeolite while stirring. The (Cs, Ga)/X material was then dried at 150° C. for 12 hours.

Deposition or addition of 1.0 wt % boron onto cesium ion-exchanged zeolite material: The cesium ion-exchanged zeolite material (35 g) was treated with a solution of boric acid (2.8 g) dissolved in acetone (500 mL) at room temperature for 2 hours. The (Cs, B)/X material was then dried at 110° C. for 20 hours. As used herein the boron content on the dried zeolite material was determined by elemental analysis unless stated otherwise.

Incipient wetness impregnation of Co(NO₃)₂ on to the cesium ion-exchanged X-zeolite material: The cesium ion-exchanged zeolite material (50 g) was subjected to incipient wetness impregnation of Co(NO₃)₂ by adding the Co(NO₃)₂ solution (2.46 g Co(NO₃)₂ in 13.3 mL of water) to the zeolite while stirring. The (Cs, Co)/X material was then dried at 150° C. for 12 hours.

Stainless steel reactor details: A stainless steel tube with 0.5-inch outer diameter and 0.465 inch internal diameter was filled with crushed quartz of 850-2000 μm size (to a height of about 10 inches, 29.2 mL), then the catalyst (to a height of 3.0 inches; 6.6 mL, 3.35 g) at sizes ranging from 250 to 425 μm, and then more crushed quartz of 850 to 2000 μm size (to a height of about 17 inches, 37.2 mL) such that a 0.125 inch stainless steel thermowell was positioned in the middle of the catalyst bed.

Ceramic lined stainless steel reactor details: Experiments were carried out with methanol and toluene over the respective catalyst. A 0.75-inch outside diameter stainless steel tube was fitted with a 0.5-inch inside diameter ceramic liner. The tube was then filled with crushed quartz (to a height of about 13.5 inches), then the catalyst (see Table 1) at sizes ranging from 250 to 425 μm, and then more crushed quartz (of a height of about 17 inches) such that a silcosteel coated thermowell was positioned in the middle of the bed. The reactor was installed in a 3-zone furnace and heated to 500° C. and held for 2 hours while passing nitrogen through it at 150 cc/min. The reactor was then cooled to the reaction temperature of 420° C. The feed was comprised of toluene, methanol and nitrogen. The flow rates were corrected for temperature, the flow rate of gases at the reaction temperature is found in Table 1 as well as the contact time. The effluent was monitored by an on-line gas chromatograph.

The information in Table 1 describes the conditions used in testing various catalysts for producing styrene and ethylbenzene from toluene and methanol:

TABLE 1

| | | | | N₂ | | | | | |
|---|---|---|---|---|---|---|---|---|---|
| | | MeOH | PhMe | (carrier | Tol/MeOH | | | Contact | Time on |
| | Catalyst | (Liq) | (Liq) | gas) | (molar | Temp | Press | Time | stream |
| Catalyst | Size | (mL/hr) | (mL/hr) | (cc/min) | ratio) | (° C.) | psig | (s) | min |
| Cs/X | 250-425 | 4.9 | 13.0 | 20 | 1.0 | 420 | 3.7 | 1.5 | 131 |
| | micron | | | | | | | | |
| Cs/X | 2 mm | 2.3 | 23.0 | 20 | 3.7 | 420 | 5 | 4.1 | 123 |
| Cs, B/X | 250-425 | 1.6 | 18.0 | 28 | 3.9 | 420 | 4 | 1.9 | 108 |
| | micron | | | | | | | | |
| Cs, B/X | 250-425 | 5.4 | 14.0 | 28 | 1.0 | 420 | 5 | 1.6 | 243 |
| | micron | | | | | | | | |
| Cs, Co/X | 250-425 | 1.5 | 16.9 | 28 | 4.3 | 420 | 2 | 1.6 | 131 |
| | micron | | | | | | | | |
| Cs, Co/X | 250-425 | 4.9 | 13.0 | 28 | 1.0 | 420 | 4 | 1.5 | 196 |
| | micron | | | | | | | | |
| Cs, Ga/X | 250-425 | 1.5 | 17.0 | 28 | 4.3 | 420 | 1.8 | 1.6 | 117 |
| | micron | | | | | | | | |
| Cs, Ga/X | 250-425 | 4.9 | 13.0 | 28 | 1.0 | 420 | 2.6 | 1.4 | 318 |
| | micron | | | | | | | | |
| (Cs, 1.0 | 250-425 | 5 | 13 | 70 | 1.0 | 420 | 1.5 | 2.6 | 95 |
| wt % B)/X | micron | | | | | | | | |

Table 2 shows the results of the experiments from Example #1 showing the toluene conversion X_{Tol} and selectivities to ethylbenzene S_{EB}, styrene S_{Sty}, benzene S_{Bz}, and xylenes S_{Xyl}. The X-zeolite based catalyst demonstrated a higher toluene conversion and high EB selectivity over the comparable other zeolite based catalysts. The (Cs, Ga)/X catalyst demonstrated a higher toluene conversion than the Cs/X and (Cs,B)/X catalysts.

TABLE 2

| | X_{Tol} | S_{EB} | S_{Sty} | | S_{Bz} | S_{Xyl} |
|---|---|---|---|---|---|---|
| Catalyst | wt % | mol % | mol % | S_{Sty}/S_{EB} | mol % | mol % |
| Cs/X | 7.2 | 83.6 | 8.2 | 0.1 | 0.25 | 0.0 |
| Cs/X | 7.5 | 82.2 | 8.7 | 0.1 | 1.4 | 0.5 |
| Cs, B/X | 10.0 | 80.3 | 11.2 | 0.1 | 0.9 | 0.0 |
| Cs, B/X | 11.5 | 77.5 | 14.2 | 0.2 | 0.4 | 0.0 |
| Cs, Co/X | 9.0 | 87.6 | 4.1 | 0.0 | 2.5 | 0.0 |
| Cs, Co/X | 12.0 | 87.5 | 3.5. | 0.0 | 3.2 | 0.0 |
| Cs, Ga/X | 3.8 | 90.9 | 2.3 | 0.0 | 1.0 | 0.0 |
| Cs, Ga/X | 14.6 | 89.1 | 4.4 | 0.0 | 0.4 | 0.0 |
| (Cs, 1.0 wt % B)/X | 18.7 | 81.0 | 16.2 | 0.2 | 0.5 | 0.1 |

### Example 2

Another experiment was carried out with 1,3,5-trioxane and toluene over Cs/X and (Cs, B)/X catalysts. A 0.75-inch diameter stainless steel tube was fitted with a 0.5-inch inside diameter ceramic liner. The tube was then filled with crushed quartz (to a height of about 6 inches) such that a silcosteel coated thermowell was positioned in the middle of the bed. The reactor was installed in a 3-zone furnace and heated to 500° C. for 6 hours while passing nitrogen through the reactor at 150 cc/min. The reactor was then cooled to the reaction temperature. The feed contained 1,3,5-trioxane dissolved in toluene (see Table 3) and nitrogen (28 cc/min). The effluent was monitored by an on-line gas chromatograph.

The Cs/X catalyst was made by the following procedure: A glass cylinder (2″ inside diameter), fitted with a sintered glass disk and stopcock at the lower end, was charged with 544-HP zeolite (100 g, W.C. Grace) and CsOH (400 mL, 1.0 M in water). The mixture was then brought to 90° C. and allowed to stand for 4 h. The liquid was drained from the zeolite material and another aliquot of CsOH (400 mL of 1.0 M solution in water) was added, heated, and allowed to stand for 3 hours at 90° C. The liquid was drained from the zeolite material and another aliquot of CsOH (400 mL of 1.0 M solution in water) was added, heated, and allowed to stand for 15 hours at 90° C. The liquid was drained from the zeolite material and dried at 150° C. for 1.5 hours.

The (Cs, B)/X catalyst was prepared by deposition of 1.0 wt % boron onto cesium ion-exchanged zeolite material: The cesium ion-exchanged zeolite material (35 g) was treated with a solution of boric acid (2.8 g) dissolved in acetone (500 mL) at room temperature for 2 hours. The (Cs, B)/X material was then dried at 110° C. for 20 hours.

The information in Table 3 describes the conditions used in testing various catalysts for producing styrene and ethylbenzene from toluene and methanol:

TABLE 3

| | | | mol % | Flow rate of | | | |
|---|---|---|---|---|---|---|---|
| | Catalyst | Reaction | trioxane | toluene + | Nitrogen | WHSV | Contact |
| Catalyst | (g) | Temp ( ° C.) | in toluene | trioxane (cc/h) | (cc/min) | (1/h) | time (s) |
| Cs/X | 11.4 | 425 | 10 | 7 | 28 | 0.5 | 5.0 |
| | | 425 | 10 | 26 | 28 | 1.8 | 2.1 |
| Cs/X | 11.4 | 425 | 22 | 6 | 28 | 0.4 | 5.0 |
| | | 425 | 22 | 25 | 28 | 1.7 | 2.0 |
| Cs/X | 11.8 | 375 | 10 | 8 | 28 | 0.5 | 5.1 |
| | | 375 | 10 | 31 | 28 | 1.9 | 2.0 |
| (Cs, B)/X | 11.2 | 425 | 10 | 7 | 28 | 0.5 | 5.0 |
| | | 425 | 10 | 26 | 28 | 1.7 | 2.1 |
| (Cs, B)/X | 11.1 | 375 | 10 | 8 | 28 | 0.5 | 5.0 |
| | | 375 | 10 | 31 | 28 | 1.9 | 2.1 |

Table 4 shows the results of the experiments, which demonstrate toluene conversion and selectivities to desired products. As used throughout all conversions and selectivity's are in mol % if not stated otherwise.

TABLE 4

| | X_{Tol} | S_{Sty} | S_{EB} | S_{Cumene} | S_{Xyl} |
|---|---|---|---|---|---|
| Catalyst | mol % | mol % | mol % | mol % | mol % |
| Cs/X | 4.6 | 9.1 | 68.3 | 6.2 | 0 |
| Cs/X | 2.6 | 5.5 | 80.3 | 3.9 | 0 |
| Cs/X | 7.2 | 1.8 | 85.6 | 3.6 | 0 |
| Cs/X | 6.5 | 12.7 | 75.4 | 3.7 | 0 |
| Cs/X | 5.0 | 19.9 | 63.2 | 8.2 | 0 |
| Cs/X | 3.8 | 57.2 | 29.2 | 6.1 | 0 |
| (Cs, B)/X | 5.2 | 3.3 | 86.6 | 1.4 | 0 |
| (Cs, B)/X | 4.5 | 21.3 | 87.5 | 2.4 | 0 |
| (Cs, B)/X | 4.9 | 15.4 | 70.9 | 4.7 | 0 |
| (Cs, B)/X | 5.7 | 62.2 | 30.0 | 2.9 | 0 |

### Example 3

Additional experimentation was carried out studying methods of introducing boron and cesium to an X-type zeolite. For baseline experimentation, boron 1.0 wt % was impregnated using anhydrous acetone as a solvent onto a Na/X zeolite to form a B/X catalyst (I) and an experiment carried out at 420° C., 2.6 second contact time, and toluene:methanol molar ratio of 1:1. As expected, the selectivity to xylenes was extremely high, see Table 5. To prepare the B/X catalyst (I) 1.52 g of boric acid was dissolved in 500 mL of acetone to form a boric acid solution. 100 g of X type zeolite (Na/X 544-HP) was added to the boric acid solution. After 2 hours, the Na/X was filtered and then transferred to a ceramic dish and placed in the hood at room temperature for 3 hours and then transferred to a oven set at 150° C. for 20 hours to dry.

Additional experimentation included preparing a second catalyst (II) by performing a cesium ion exchange on the boron-impregnated B/X catalyst (I). The second catalyst (II) was prepared with the following procedure: 50 g of boron deposited zeolite (B/X catalyst (I)) was placed in a ion exchange column along with 400 mL of 1M CsOH. A thermocouple was secured to the side of the column with heat tape set at 90° C. in the area where the material was placed. After 4 hours the liquid was drained from the column and an additional 400 mL of 1.0M CsOH was added to the column, after 4 hours it was again drained. A third additional 400 mL of 1.0M CsOH was added and the ion exchange column was kept at 90° C. for a period of 16 hours. The material was then filtered and dried in a static drying oven at 150° C. for 20 hours.

Additional experimentation included preparing a third catalyst (III) by performing a cesium ion exchange on the Na/X and then the boron is placed in the zeolite by impregnation. The third catalyst (III) was prepared with the following procedure: A solution of CsOH (1 L; 1 M; 165.73 g) was prepared in distilled water. 100 g of zeolite (Na/X) was added to a round bottom flask along with 400 mL of 1M cesium hydroxide solution. The flask was heated in an oil bath set at 90° C. for a first exchange. After 16 hours the liquid was drained and an additional 400 mL of 1.0M CsOH was added and kept at 90° for 4 hours for a second exchange. After the second exchange the liquid was drained and an additional 400 mL of 1.0M CsOH was added and kept at 90° C. for 4 hours for a third exchange. After the third exchange the liquid was drained and the material was allowed to dry at ambient temperature for 3 hours then 20 hours in a drying oven at 150° C. For the boron addition to the Cs/X, 1.52 g of boric acid was dissolved in 500 mL of acetone to form a boric acid solution. 100 g of Cs/X zeolite was added to the boric acid solution. After 2 hours, the Cs/X zeolite was filtered and then transferred to a ceramic dish and placed in the hood at room temperature for 3 hours and then transferred to an oven set at 150° C. to dry for 20 hours.

Under the same experimental conditions, the results of catalyst (II) are similar to that found where the Cs is first introduced to the zeolite by ion-exchange and then the boron is placed in the zeolite by impregnation indicated as catalyst (III). It was found that the stability of the catalyst is enhanced by the B-impregnation/Cs-ion-exchange method over the Cs-ion exchange/B-impregnation method as indicated by the results shown in Table 5.

Also in this experiment, a catalyst (IV) was prepared to determine if the introduction of boron by aqueous means before the introduction of cesium results in different catalyst behavior from that where cesium ion-exchange is followed by boron impregnation as in catalyst (III). The catalyst (IV) was prepared with the following procedure: a solution of boric acid (1.52 g of boric acid) diluted to 500 cc with distilled water. 400 ml of the diluted boric acid was added to an ion-exchange column with 100 g of X-zeolite and kept at ambient temperature for 2 hours. The resultant material was then dried for 20 hours in a drying oven at 110° C. 100 g of the dried material was added to a round bottom flask along with 400 mL of 1M cesium hydroxide solution. The flask was heated in an oil bath set at 90° C. for a first exchange. After 16 hours the liquid was drained and an additional 400 mL of 1.0M CsOH was added and kept at 90° for 4 hours for a second exchange. After the second exchange the liquid was drained and an additional 400 mL of 1.0M CsOH was added and kept at 90° C. for 4 hours for a third exchange. After the third exchange the liquid was drained and the material was allowed to dry at ambient temperature for 3 hours then 20 hours in a drying oven at 150° C.

The catalysts were used in an alkylation of toluene and methanol (ATM) process having a toluene:methanol ratio of 1:1 under a temperature of 420° C. with a contact time of 2.5 seconds. The ATM experiments showed no appreciable catalyst deactivation over the course of the runs for catalysts (II)-(IV) and higher toluene conversion as compared to catalyst (I). Results of these experiments are shown in Table 5.

Also in this experiment, a catalyst (V) was prepared in which cesium and boron were simultaneously added and then used in the alkylation of toluene with methanol (ATM). The catalyst (V) was prepared with the following procedure: The solution for co-exchange was prepared by dissolving 3.0 g of boric acid to 1000 mL of 1.0M CsOH. 100 g of Na/X (544-HP) was placed in an ion exchange column. The ion exchange column was filled with 400 mL of the solution for co-exchange and a thermocouple placed outside the ion exchange column and was heated to 90° C. and was kept for a period of 16 hours, after which liquid from the column was drained and a sample was collected for ICP analysis. An additional 400 mL of Cs, B solution was refilled and the ion exchange column was kept at 90° C. for 4 hours in a second exchange. After the second exchange a sample was collected for ICP analysis and fresh solution for co-exchange was refilled for a third exchange at 90° C. for 4 hours. After the third exchange was complete the liquid was drained and the remaining catalyst was transferred into a ceramic dish and dried in a drying oven at 150° C. for 20 hours.

The ATM process used a toluene:methanol ratio of 1:1 under a temperature of 420° C. with a contact time of 2.7 seconds. It was found that co-exchanged B/Cs catalyst results in a lower toluene conversion (about 10 vs. about 16 mol %), similar selectivity to ethylbenzene and styrene, but a higher selectivity to xylenes (0.4 vs. 0.2 mol %) than a catalyst prepared by cesium-ion-exchange followed by boron impregnation. It appears that the catalyst stability enhancement is retained through placement of the boron before or during the addition of cesium as opposed to adding boron after cesium has been placed in the zeolite. The stability being the deactivation rate as a difference in toluene conversion over time. Results are shown in Table 5.

TABLE 5

| | | Time On | | | | | |
|---|---|---|---|---|---|---|---|
| | | Stream | | | | | |
| | Catalyst | (hh:mm) | X_{Tol} | S_{Bz} | S_{Xyl} | S_{EB} | S_{Sty} |
| B-impregnated | (I) | 0:33 | 7.2 | 0.9 | 91.7 | 1.1 | 0.1 |
| onto NaX | B/X | | | | | | |
| | | 3:43 | 1.5 | 0.9 | 85.4 | 3.8 | 1.8 |
| | | 5:06 | 0.4 | 1.8 | 91.9 | 2.8 | 0.8 |
| B impregnation, | (II) | 1:30 | 12.4 | 0.3 | 0.1 | 92.1 | 6.3 |
| then Cs-ion- | B, Cs/X | | | | | | |
| exchange | | | | | | | |
| | | 2:20 | 12.2 | 0.3 | 0.2 | 92.0 | 6.5 |
| | | 3:15 | 12.3 | 0.3 | 0.2 | 92.2 | 6.5 |
| | | 4:25 | 12.3 | 0.2 | 0.2 | 92.4 | 6.4 |
| Cs-ion-exchange | (III) | 1:35 | 19 | 0.5 | 0.1 | 81.0 | 16.2 |
| then B | Cs, B/X | | | | | | |
| impregnation | | | | | | | |
| | | 2:25 | 17 | 0.4 | 0.1 | 81.8 | 15.5 |
| | | 3:25 | 17 | 0.3 | 0.1 | 82.4 | 15.1 |
| | | 4:25 | 15 | 0.3 | 0.1 | 82.5 | 15 |
| B aqueous | (IV) | 0:44 | 15.0 | 0.4 | 0.2 | 96.5 | 2.6 |
| addition, then | B, Cs/X | | | | | | |
| Cs-ion-exchange | | | | | | | |
| | | 2:04 | 17.3 | 0.3 | 0.1 | 95.1 | 3.9 |
| | | 3:29 | 16.7 | 0.2 | 0.2 | 95.0 | 4.1 |
| | | 5:24 | 16.3 | 0.2 | 0.2 | 95.0 | 4.1 |
| Cs-ion-exchange | (V) | 1:00 | 8.8 | 0.6 | 0.4 | 96.2 | 2.3 |
| and B | Cs, B/X | | | | | | |
| impregnation | | | | | | | |
| at same time | | | | | | | |
| | | 2:00 | 10.1 | 0.5 | 0.4 | 96.2 | 2.6 |
| | | 3:15 | 10.5 | 0.4 | 0.4 | 95.6 | 3.3 |

A rough estimate of the results, depicted in Table 6, as a function of how the boron and cesium were placed in the zeolite shows that all four routes give reasonable results in ATM experiments.

TABLE 6

| | X_{Tol} | S_{Bz} | S_{Xyl} | S_{EB} | S_{Sty} |
|---|---|---|---|---|---|
| Catalyst (III) | 17 | 0.3 | <0.2 | 82 | 15 |
| Cs ion-exchange, then B impregnation | | | | | |
| Catalyst (IV) | 16 | 0.3 | <0.2 | 95 | 4 |
| B aqueous addition, then Cs | | | | | |
| ion-exchange | | | | | |
| Catalyst (II) | 12 | 0.3 | <0.2 | 92 | 6 |
| B impregnation, then Cs ion-exchange | | | | | |
| Catalyst (V) | 10 | 0.5 | 0.4 | 96 | 3 |
| B and Cs added at same time | | | | | |

### Example 4

Additional experimentation was performed in order to improve both methanol and toluene conversion and selectivity to the desired products. In pursuit of these ends, the optimal method of boron introduction as well as boron concentration in an X-zeolite was studied. In this experiment, three additional catalysts were prepared with boric acid such that they contain 1, 2 and 3 wt % boron. These catalysts were used in ATM experiments for comparison to that of a catalyst having 0.3 wt. % boron. All experiments were carried out at 420° C.

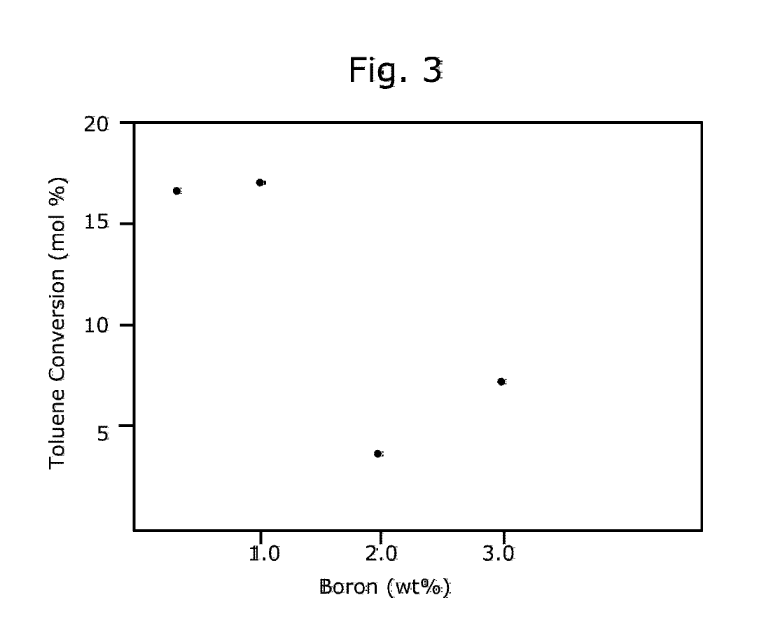

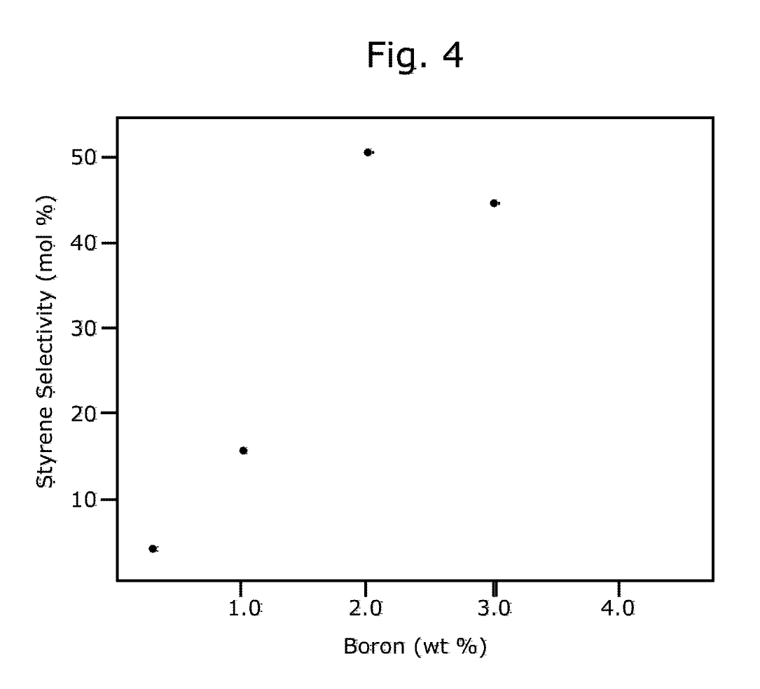

The results show that 1 wt % boron achieved the best results for toluene conversion, followed by 0.3 wt % boron, then 2 wt % boron, followed finally by 3 wt % boron (see FIG. 3 showing data at approx. 3.5 hr runtime). The results show that boron concentration also has an effect on styrene selectivity. The results indicate that styrene selectivity increases as the boron concentration increases and at a point between 1 wt % boron and 2 wt % boron a large increase, of about 35%, in styrene selectivity occurs (See FIG. 4 showing data at approx. 3.5 hr runtime). Furthermore, the results show that by decreasing the relative amount of methanol (moving from 1:1 To 4.1:1 molar ratio of toluene to methanol) it was found that the toluene conversion decreased and the selectivity to styrene over ethylbenzene was enhanced. Thus, it appears that the judicious introduction of cesium and boron will allow the separate maximization of the side-chain alkylation and minimize the decomposition of methanol/formaldehyde. The results of this experiment are depicted in Table 7.

TABLE 7

| | Tol:MeOH | | Contact | Time On | | | | | |
|---|---|---|---|---|---|---|---|---|---|
| | (molar | LHSV | Time | Stream | | | | | |
| Catalyst | ratio) | (h⁻¹) | (s) | (hh:mm) | X_{Tol} | S_{Bz} | S_{Xyl} | S_{EB} | S_{Sty} |
| | | | | | By C in Effluent | | | | |
| (Cs, B)/X: 0.3 wt % B | 1.0 | 1.6 | 2.7 | 2:04 | 17.3 | 0.3 | 0.1 | 95.1 | 3.9 |
| | | | | 3:29 | 16.7 | 0.2 | 0.2 | 95.0 | 4.1 |
| | | | | 5:24 | 16.3 | 0.2 | 0.2 | 95.0 | 4.1 |
| (Cs, B)/X: 1 wt % B | 1.0 | 1.2 | 2.4 | 1:35 | 18.7 | 0.5 | 0.1 | 81.0 | 16.2 |
| | | | | 2:25 | 17.2 | 0.4 | 0.1 | 81.8 | 15.5 |
| | | | | 3:25 | 17.0 | 0.3 | 0.1 | 82.4 | 15.1 |
| | | | | 4:25 | 15.3 | 0.3 | 0.1 | 82.5 | 15.0 |
| (Cs, B)/X: 2 wt % B | 1.0 | 1.5 | 2.5 | 2:07 | 7.9 | 0.8 | 0.1 | 56.6 | 40.7 |
| | 4.1 | 2.0 | | 3:17 | 3.9 | 1.0 | 0.2 | 47.0 | 50.3 |
| | | | | 4:37 | 3.2 | 0.9 | 0.3 | 46.1 | 51.7 |
| (Cs, B)/X: 3 wt % B | 1.0 | 1.4 | 2.4 | 1:10 | 9.9 | 1.1 | 0.1 | 58.5 | 38.4 |
| | | | | 2:15 | 9.1 | 0.7 | 0.2 | 52.2 | 44.9 |
| | | | | 3:20 | 7.6 | 0.7 | 0.2 | 52.3 | 45.0 |
| | | | | 4:30 | 6.8 | 0.6 | 0.2 | 52.6 | 44.5 |
| | | | | 7:30 | 2.4 | 0.6 | 0.3 | 35.9 | 62.0 |
| | 3.8 | 2.5 | 1.8 | 8:25 | 2.4 | 0.6 | 0.4 | 36.4 | 61.6 |

### Example 5

Further experimentation was performed to determine if alternatives to known boron sources, such as boric acid and boron phosphate, could be utilized in efficiently masking the overly basic sites of the X-type zeolite. In this experiment, catalysts were obtained by impregnating a zeolite catalyst using boroxines as the boron source. The catalysts were prepared by impregnating a Cs/X catalyst with 1 wt % boron via methoxyboroxine or methylboroxine. The ATM experiments were carried out at 420° C., with a 2.5 second contact time, and a 1:1 molar ratio of toluene to methanol.

The catalyst made from methoxyboroxine was prepared with the following procedure: A solution of CsOH (1 L; 1 M; 165.73 g) was prepared in distilled water. 100 g of zeolite (Na/X) was added to a round bottom flask along with 400 mL of 1M cesium hydroxide solution. The flask was heated in an oil bath set at 90° C. for a first exchange. After 16 hours the liquid was drained and an additional 400 mL of 1.0M CsOH was added and kept at 90° C. for 4 hours for a second exchange. After the second exchange the liquid was drained and an additional 400 mL of 1.0M CsOH was added and kept at 90° C. for 4 hours for a third exchange. After the third exchange the liquid was drained and the material was allowed to dry at ambient temperature for 3 hours then 20 hours in a drying oven at 150° C. to form a (Cs, Na)/X zeolite. 15 mL of acetone and 2.20 mL of tri methyloxy boroxine were combined to made a homogeneous mixture, to which 50.0 g of (Cs, Na)/X zeolite was added and stirred to dryness at room temperature. The catalyst was then dried at 75° C. for 4 hours.

The catalyst made from methylboroxine was prepared with the same procedure as above but using 15 mL of acetone and 2.14 mL of trimethyl boroxine to make the homogeneous mixture to which 50.0 g of (Cs, Na)/X zeolite was added and stirred to dryness at room temperature. The catalyst was then dried at 75° C. for 4 hours.

For the methoxyboroxine based catalyst, the initial sample held approximately the same toluene conversion as when boric acid based catalyst was used. However, this conversion dropped substantially after about 3 hours on stream. Both uses of boroxines result in lower toluene conversion and faster catalyst deactivation. However, the selectivity to styrene was noticeably enhanced by the catalysts made from the boroxines. The results indicate, that the use of boroxines as the boron source can increase styrene selectivity by up to 35%. Table 8 compares the catalysts prepared by boroxines with the catalyst prepared by boric acid.

TABLE 8

| | | Time On | | | | | |
|---|---|---|---|---|---|---|---|
| | Boron | Stream | | | | | |
| Catalyst | Source | (hh:mm) | X_{Tol} | S_{Bz} | S_{Xyl} | S_{EB} | S_{Sty} |
| | | | By C in Effluent | | | | |
| (Cs, B)/X | (BO(OMe))₃ | 1:45 | 15 | 0.6 | 0.1 | 70.5 | 26.4 |
| | | 2:55 | 11 | 0.6 | 0.1 | 67.8 | 29.3 |
| | | 1:40 | 11 | 0.9 | 0.2 | 48.4 | 50.5 |
| (Cs, B)/X | (B(O)(Me))₃ | 3:15 | 8 | 0.8 | 0.2 | 51.7 | 47.3 |
| | | 5:45 | 5 | 0.8 | 0.3 | 57.2 | 41.6 |
| | | 1:35 | 19 | 0.5 | 0.1 | 81 | 16.2 |
| (Cs, B)/X | B(OH)₃ | 2:25 | 17 | 0.4 | 0.1 | 81.8 | 15.5 |
| | | 3:25 | 17 | 0.3 | 0.1 | 82.4 | 15.1 |
| | | 4:25 | 15 | 0.3 | 0.1 | 82.5 | 15 |

The term “conversion” refers to the percentage of reactant (e.g. toluene) that undergoes a chemical reaction.

X_{Tol}=conversion of toluene (mol %)=(Tolᵢₙ−Tolₒᵤₜ)/Tolᵢₙ

X_{MeOH}=conversion of methanol to styrene+ethylbenzene (mol %)

The term “molecular sieve” refers to a material having a fixed, open-network structure, usually crystalline, that may be used to separate hydrocarbons or other mixtures by selective occlusion of one or more of the constituents, or may be used as a catalyst in a catalytic conversion process.

Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

The term “regenerated catalyst” refers to a catalyst that has regained enough activity to be efficient in a specified process. Such efficiency is determined by individual process parameters.

The term “selectivity” refers to the relative activity of a catalyst in reference to a particular compound in a mixture. Selectivity is quantified as the proportion of a particular product relative to all other products.

S_{Sty}=selectivity of toluene to styrene (mol %)=Styₒᵤₜ/Tol_{converted}

S_{Bz}=selectivity of toluene to benzene (mol %)=Benzeneₒᵤₜ/Tol_{converted}

S_{EB}=selectivity of toluene to ethylbenzene (mol %)=EBₒᵤₜ/Tol_{converted}

S_{Xyl}=selectivity of toluene to xylenes (mol %)=Xylenesₒᵤₜ/Tol_{converted}

S_{Sty+EB} (MeOH)=selectivity of methanol to styrene+ethylbenzene (mol %)=(Styₒᵤₜ+EBₒᵤₜ)/MeOH_{converted}

The term “spent catalyst” refers to a catalyst that has lost enough catalyst activity to no longer be efficient in a specified process. Such efficiency is determined by individual process parameters.

The term “zeolite” refers to a molecular sieve containing an aluminosilicate lattice, usually in association with some aluminum, boron, gallium, iron, and/or titanium, for example. In the following discussion and throughout this disclosure, the terms molecular sieve and zeolite will be used more or less interchangeably. One skilled in the art will recognize that the teachings relating to zeolites are also applicable to the more general class of materials called molecular sieves. An X-zeolite is defined as having a Si/Al molar ratio between 1.0 and 1.5. A Y-zeolite is defined as having a Si/Al molar ratio greater than 1.5.

The various aspects of the present invention can be joined in combination with other aspects of the invention and the listed embodiments herein are not meant to limit the invention. All combinations of various aspects of the invention are enabled, even if not given in a particular example herein.

Depending on the context, all references herein to the “invention” may in some cases refer to certain specific embodiments only. In other cases it may refer to subject matter recited in one or more, but not necessarily all, of the claims. While the foregoing is directed to embodiments, versions and examples of the present invention, which are included to enable a person of ordinary skill in the art to make and use the inventions when the information in this patent is combined with available information and technology, the inventions are not limited to only these particular embodiments, versions and examples. Other and further embodiments, versions and examples of the invention may be devised without departing from the basic scope thereof and the scope thereof is determined by the claims that follow.

What is claimed is:

1. A method of preparing a catalyst, comprising:
providing a substrate;
providing a first solution comprising at least one promoter; and
contacting the substrate with the first solution; and
obtaining a catalyst comprising at least one promoter;
wherein the contacting of the substrate with the solution subjects the substrate to the addition of at least one promoter.

2. The method of claim 1, wherein the substrate is a zeolite.

3. The method of claim 1, wherein the at least one promoter is selected from the group consisting of Ru, Rh, Ni, Co, Pd, Pt, Mn, Ti, Zr, V, Nb, K, Cs, Ga, B, P, Rb, Ag, Na, Cu, Mg, and combinations thereof.

4. The method of claim 1, wherein the at least one promoter is selected from the group consisting of K, Cs, Ga, Rb, and B and combinations thereof.

5. The method of claim 1, further comprising a second solution comprising Cs, wherein the at least one promoter of the first solution comprises B and wherein the first and second solution simultaneously contact the substrate resulting in simultaneous addition of the B and Cs to the substrate, resulting in a substrate comprising B and Cs.

6. The method of claim 1, further comprising a second solution comprising Cs, wherein the at least one promoter of the first solution comprises B and wherein the first solution initially contacts the substrate resulting in the addition of B resulting in a substrate comprising B, followed by contacting the substrate comprising B with the second solution comprising Cs resulting in ion exchange between the cationic sites on the substrate and the Cs finally resulting in a substrate comprising B and Cs.

7. The method of claim 1, wherein the catalyst comprises B in amounts ranging from 0.1 wt % to 3 wt % based on the total weight of the catalyst.

8. The method of claim 5, wherein the B in the first solution is supplied by a boron source comprising boroxines.

9. The method of claim 1, wherein the catalyst is capable of effecting a reaction of at least a portion of a C₁ source with toluene to form a product stream comprising one or more of styrene or ethylbenzene.

10. The method of claim 9, wherein a boron source is combined with the C₁ source prior to contact with the catalyst.

11. The method of claim 1, wherein a boron source is combined with the substrate prior to contacting the substrate with the first solution.

12. The method of claim 1, wherein a boron source is combined with a substrate material that is subsequently combined with the catalyst comprising at least one promoter to form a supported catalyst comprising at least one promoter.

13. A catalyst comprising:
a zeolitic support;
at least one promoter selected from the group consisting of Cs, B, Ga, Rb, and K, and combinations thereof;
wherein at least one promoter is supported onto the zeolitic support by ion exchange.

14. The catalyst of claim 13, wherein the at least one promoter contains B obtained from a boron source.

15. The catalyst of claim 14, wherein the boron source comprises boroxines.

16. The catalyst of claim 13, wherein the at least one promoter is a combination of Cs and B.

17. The catalyst of claim 14, wherein the boron is present in the catalyst in amounts of from 0.1 to 3 wt % based on the total weight of the catalyst.

18. The catalyst of claim 13, wherein the ion exchange is performed in an aqueous medium utilizing water soluble promoter precursors.

19. The catalyst of claim 13, wherein a boron source is combined with a substrate material that is subsequently combined with the zeolitic support comprising at least one promoter to form a supported catalyst comprising at least one promoter.

20. The catalyst of claim 13, wherein the catalyst is capable of effecting a reaction of at least a portion of a C₁ source with toluene to form a product stream comprising one or more of styrene or ethylbenzene, wherein the catalyst is capable of effecting selectivity to styrene of greater than 30 mol %.

21. A process for making styrene comprising:
providing a C₁ source to a reactor comprising a catalyst; and
reacting toluene with the C₁ source in the presence of the catalyst to form a product stream comprising ethylbenzene and styrene;
wherein the C₁ source is selected from the group consisting of methanol, formaldehyde, formalin, trioxane, methylformcel, paraformaldehyde, methylal, and combinations thereof;
wherein the catalyst comprises B and Cs supported on a zeolite.

22. The process of claim 21, wherein the B and Cs were added to the zeolite in an aqueous medium utilizing water-soluble B and Cs precursors.

23. The process of claim 21, wherein a boron source is added to the C₁ source and/or the toluene feed.

24. The process of claim 21, wherein the catalyst is a supported catalyst made from a boron source combined with a substrate material that is added to the catalyst that has B and Cs supported on a zeolite.
